(19) United States
(12) Patent Application Publication
(10) Pub. No.: US 2014/0051640 A1
(43) Pub. Date: Feb. 20, 2014

(54) PLANT PROTEIN AND MINERAL POWDER-BASED COLLAGEN MASK

(71) Applicant: Li YANG, Xi'an (CN)

(72) Inventor: Li YANG, Xi'an (CN)

(21) Appl. No.: 13/953,763

(22) Filed: Jul. 30, 2013

Related U.S. Application Data
(60) Provisional application No. 61/742,626, filed on Aug. 15, 2012.

(51) Int. Cl. A61K 8/64; A61Q 19/00

(52) U.S. Cl. CPC A61K 8/645; A61Q 19/007
USPC 514/18.8

(57) ABSTRACT

The present patent application relates to a plant protein and mineral powder-based collagen mask, comprising the following constituents by weight percentage: Soya Protein: 1.2-1.5; Ethylene Glycol Phenyl Ether: 0.2-0.5; the rest is purified water. The plant protein and mineral powder-based collagen mask produced by the formula of the present patent application has a strong hydrating function. The moisture content of the plant protein and mineral powder-based collagen mask can be as high as 90%. When meet 37-38 degree body temperature, the plant protein and mineral powder-based collagen mask will release moisture slowly and fully releases after 4-5 hours, so that the skin always keep being moist. Meanwhile, the plant protein and mineral powder-based collagen mask of the present patent application will not suck back the moisture of the skin.

### FIELD OF PATENT APPLICATION

The present patent application relates to the field of cosmetic, especially relates to a plant protein and mineral powder-based collagen mask.

### BACKGROUND

At present, the crystal membrane includes facial mask, eye mask, lip mask, eye socket mask, neck mask, breast mask, hand mask etc. Take facial mask as example, the crystal membrane on the market is called wax facial mask. When using, the cosmetic membrane is heated and then smeared on the face after melting. The surface of the skin is warmed and the ability of absorbing the nutrition of the skin is increased. Then the nutrient substance permeates into the skin. Meanwhile, the sealing property of the cosmetic wax is very good and the moisture content of the stratum corneum of the skin can be locked. But when such a facial mask use the sweat excreted from the body to increase the moisture content of the stratum corneum of the skin, the waste and the saline of the sweat come back to the stratum corneum with the moisture due to they can't evaporate. Although the skin looks soft and moist at that time, it will be hurt by long-term use. The other existing cosmetic membranes on the market have less efficiency in moisturizing and preserving moisture and the skin can't be ameliorated substantially.

### SUMMARY

The present patent application solves the above technical problem by providing a plant protein and mineral powder-based collagen mask. The plant protein and mineral powder-based collagen mask can cause a qualitative change to the skin and achieve a good moisturizing effect, and have no harm to the user's skin.

The plant protein and mineral powder-based collagen mask of the present patent application comprises the following constituents by weight percentage: Soya Protein: 1.2-1.5; Ethylene Glycol Phenyl Ether: 0.2-0.5; the rest is purified water.

According to one aspect of the present patent application, the plant protein and mineral powder-based collagen mask further comprises moisturizing ingredients. The moisturizing ingredients comprising the following constituents by weight percentage: Animo Acid Moisturiser: 1-3; Allantoin: 0.2-0.5; Pumice Stone Powder: 2-5; 1.2-Propanediol: 1-4; Glycerin: 1-4; Hyaluronic Acid: 0.02-1.

According to another aspect of the present patent application, the plant protein and mineral powder-based collagen mask further comprises the floral essential oil by weight percentage of 0.05-0.1. The floral essential oil consists of one or any combination of two of rose essential oil, lotus extract, lemon essential oil, Lavender Essential Oil, Seaweed Trehalose, Orange Extract, Matricaria Recutica (Chamomile), tea tree essential oil and jasmine essential oil.

According to another aspect of the present patent application, the plant protein and mineral powder-based collagen mask further comprises an additive by weight percentage of 0.1, the additive is one or combination of phlogopite and gold leaf.

The plant protein and mineral powder-based collagen mask produced by the formula of the present patent application has a strong hydrating function. The moisture content of the plant protein and mineral powder-based collagen mask can be as high as 90%. When meet 37-38 degree body temperature, the plant protein and mineral powder-based collagen mask will release moisture slowly and fully releases after 4-5 hours, so that the skin always keep being moist. The plant protein and mineral powder-based collagen mask has very good hydrating effect especially for frostbite skin, sun burned skin and very dry skin. Meanwhile, the plant protein and mineral powder-based collagen mask of the present patent application will not suck back the moisture of the skin. The plant protein and mineral powder-based collagen mask of present patent application will decompose automatically when qualitative change happens or the bacteria content exceeds a criterion, so that it cannot be used by users. So that Thus the skin of the users won't have allergies or be hurt.

### BRIEF DESCRIPTION OF THE DRAWINGS

FIG. 1 is the process flow chart of the present patent application.

### DETAILED DESCRIPTION

The present patent application will be further described with the following embodiments, but the following embodiments shall not be regarded as limiting of the present patent application.

### Embodiment 1

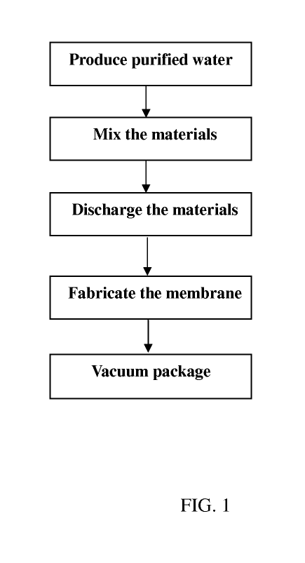

FIG. 1 is the process flow chart of the present patent application. The first step is producing water. The water should be purified ionized water and disinfected before using.

Then boil the purified ionized water, put in the constituents of the plant protein and mineral powder-based collagen mask and mix the materials. Stir for 45-70 minutes and then discharge the materials when the temperature of the slurry reaches 60-75 degrees. The constituents by weight percentage of the plant protein and mineral powder-based collagen mask are: Soya Protein: 1.4; p Ethylene Glycol Phenyl Ether: 0.25; Animo Acid Moisturiser: 1; Allantoin: 0.25; Pumice Stone Powder: 3.5; 1.2-Propanediol: 2.5; Glycerin: 2; Hyaluronic Acid: 0.03; Rose Oil 0.05; Phlogopite 0.08; gold leaf 0.02; purified water 88.92.

Then the discharged materials are poured into a pre-prepared PVC mould and quickly frozen to room temperature. The product will be placed into a bag and be sealed through vacuum sealing by the line operator.

### Embodiment 2

The operational method of the embodiments 2 is the same as embodiments 1 except the constituents of the plant protein and mineral powder-based collagen mask. The constituents by weight percentage of embodiments 2 are as following: Soya Protein: 1.5; Ethylene Glycol Phenyl Ether: 0.2; Animo Acid Moisturiser: 1.2; allantoin: 0.3; Pumice Stone Powder: 3; 1.2-Propanediol: 3; Glycerin: 2; Hyaluronic Acid: 0.05; lotus extract 0.05; purified water 88.7.

### Embodiment 3-10

The operational methods of the embodiments 3-10 are the same as embodiments 1 except the constituents of the plant protein and mineral powder-based collagen mask. The constituents by weight percentage of embodiments 3-10 are shown in below table:

| | Weight (%) | | | | | | | |
|---|---|---|---|---|---|---|---|---|
| | Embodiment | Embodiment | Embodiment | | | | | Embodiment |
| Name | 3 | 4 | 5 | Embodiment 6 | Embodiment 7 | Embodiment 8 | Embodiment 9 | 10 |
| Soya Protein | 1.2 | 1.3 | 1.25 | 1.4 | 1.35 | 1.5 | 1.4 | 1.4 |
| Ethylene Glycol Phenyl | 0.3 | 0.4 | 0.2 | 0.3 | 0.5 | 0.2 | 0.25 | 0.3 |
| Ether | | | | | | | | |
| Animo Acid Moisturiser | 2 | 1 | 1 | 1.5 | 1 | 1 | 3 | 1.5 |
| Allantoin | 0.25 | 0.2 | 0.3 | 0.25 | 0.25 | 0.4 | 0.5 | 0.25 |
| Pumice Stone Powder | 3.5 | 3 | 5 | 4 | 3.5 | 2 | 3 | 3.5 |
| 1.2-Propanediol | 2.5 | 2.5 | 2.5 | 3 | 4 | 2.5 | 2.5 | 1 |
| Glycerin | 3 | 2 | 1 | 3 | 2 | 2 | 3 | 4 |
| Hyaluronic Acid | 0.03 | 0.05 | 0.05 | 0.03 | 0.05 | 0.05 | 0.08 | 0.1 |
| Rose Essential Oil | 0.05 | | | | | | | |
| Lotus Extract | | | | | | | | 0.05 |
| Lemon Essential Oil | 0.05 | | | | | | | |
| Lavender Essential Oil | | 0.05 | | | | | | |
| Seaweed Trehalose | | | | 0.05 | | | | |
| Orange Extract | | | 0.05 | 0.05 | 0.05 | | 0.03 | |
| Matricaria Recutica | | | 0.05 | | | | | |
| (Chamomile) | | | | | | | | |
| Tea Tree Essential Oil | | | | | 0.05 | | 0.05 | |
| Jasmine Essential Oil | | | | | | 0.05 | | |
| purified water | 87.12 | 89.45 | 88.86 | 86.42 | 87.25 | 90.3 | 86.19 | 87.9 |

The plant protein and mineral powder-based collagen mask of the above embodiments was tested by using in the face and other parts of body of 330 volunteers. The plant protein and mineral powder-based collagen mask produces remarkable effect. Each volunteer uses twice each week and 30 minutes each time. After four weeks using, the skin has no allergies and become moister and more elastic.

Although the patent application has been described with respect to certain embodiments, the description is not regarded as limiting of the patent application. The alternative changes or modifications of aspects of the embodiments of the patent application fall within the spirit of the present patent application.

What's claimed is:

1. A plant protein and mineral powder-based collagen mask, wherein comprising the following constituents by weight percentage: Soya Protein: 1.2-1.5; Ethylene Glycol Phenyl Ether: 0.2-0.5; the rest is purified water.

2. The plant protein and mineral powder-based collagen mask in claim 1, wherein the crystal membrane further including comprising moisturizing ingredients.

3. The plant protein and mineral powder-based collagen mask in claim 2, wherein the moisturizing ingredients comprising the following constituents by weight percentage: Animo Acid Moisturiser: 1-3; Allantoin: 0.2-0.5; Pumice Stone Powder: 2-5; 1.2-Propanediol: 1-4; Glycerin: 1-4; Hyaluronic Acid: 0.02-1.

4. The plant protein and mineral powder-based collagen mask in claim 3, wherein the crystal membrane further including comprising the floral essential oil 0.05-0.1 by weight percentage of 0.05-0.1.

5. The plant protein and mineral powder-based collagen mask in claim 4, wherein the floral essential oil consists of one or any combination of two of rose essential oil, lotus extract, lemon essential oil, Lavender Essential Oil, Seaweed Trehalose, Orange Extract, Matricaria Recutica (Chamomile), tea tree essential oil and jasmine essential oil.

6. The plant protein and mineral powder-based collagen mask in claim 5, wherein the crystal membrane further comprising an additive by weight percentage of 0.1, the additive is one or combination of phlogopite and gold leaf.
